(19) United States
(12) Patent Application Publication
(10) Pub. No.: US 2005/0255161 A1
(43) Pub. Date: Nov. 17, 2005

(54) Method for the long term stabilization of labile compounds at room temperature in pharmaceutical preparations containing water

(76) Inventors: Servet Buyuktimkin, Robbinsville, NJ (US); Nadir Buyuktimkin, Robbinsville, NJ (US); James L. Yeager, Lake Forest, IL (US)

Correspondence Address: WELSH & KATZ, LTD, 120 S RIVERSIDE PLAZA, 22ND FLOOR, CHICAGO, IL, 60606, US

(21) Appl. No.: 11/125,545

(22) Filed: May 9, 2005

Related U.S. Application Data
(60) Provisional application No. 60/569,544, filed on May 10, 2004.

(51) Int. Cl. A61K 9/24; A61K 9/14

(52) U.S. Cl. 424/486

(57) ABSTRACT

A stabilized pharmaceutical composition that comprises a pharmaceutical compound that is labile to degradation in the presence of water is disclosed. That composition contains water, an effective amount of labile pharmaceutical compound and a degradation-inhibiting amount of a C₄-C₁₂ polyol that is substantially free of aldehyde or ketone carbonyl groups. The composition exhibits no more than 10 percent degradation of the labile pharmaceutical compound over a period of storage of at least 12 months when stored at ambient room temperature. A method of making the stabilized composition is also disclosed.

### CROSS-REFERENCE TO RELATED APPLICATION

This application claims the benefit of the filing date of U.S. Application Ser. No. 60/569,544, filed May 10, 2004.

### TECHNICAL FIELD

The present invention relates to the stabilization of labile pharmaceutical compounds in aqueous medium and encompasses compositions that are liquid, semi-solid or solid. More particularly, the present invention relates to the use of a low molecular weight carbonyl group-free polyol to stabilize aqueous compositions of labile pharmaceutical or cosmetic ingredients.

### BACKGROUND OF THE INVENTION

The long term stability of many pharmaceutical or cosmetic or cosmeceutical active ingredient(s) in a formulation is a major technical problem to overcome in order for the formulation to have utility and to be commercially viable. A cosmeceutical is a cosmetic product claimed to have medicinal or drug-like benefits; i.e., a cosmeceutical product is marketed as cosmetic, but contains biologically active ingredients. Stability of the active ingredient in a formulation is essential for maintaining proper pharmacological activity or biological function. However, it is important that stabilization of the active ingredient does not impede or significantly reduce the ability of the active ingredient to be released from the matrix of the formulation so that the intended action is achieved. To withstand the regulatory review process, formulations must be both physically and chemically stable for an adequate period of time. Pharmaceutical formulations are under strict scrutiny to maintain no less than 90% of the active ingredient throughout the intended shelf life of the product—usually 2 years—while also maintaining adequate functionality or therapeutic effect.

Several approaches have been used to stabilize drugs in water-containing medium but these approaches of stabilization have had limited success in achieving sufficient stability for regulatory approval and/or a commercially viable product. One approach presented is the temporary protection or blocking of the chemically reactive functional groups on a particular active ingredient. To this end, researchers have developed a number of protecting groups. The vast majority of the known protecting groups are designed to be cleaved in aqueous media by means of acid or base catalysis or transformed in contact with biological medium into active material. However these compounds containing labile protective group(s) are treated by the regulatory agencies as new chemical entities and are subject to extensive amount of very expensive toxicity, irritation and clinical testing. Addition of protective groups as a means to improve stability is a very costly endeavor due to the additional development and regulatory cost.

It is also possible to formulate compositions in a different medium where the active material is stable, thereby avoiding the source of the instability, however it is well known that such an approach generally can lead to a formulation with strongly reduced therapeutic activity. Therefore it is imperative to develop approaches to stabilize the formulation in an appropriate medium in order to facilitate, enhance, enable or allow a therapeutic modality or improved patient compliance and shelf-life without affecting its activity.

It is also common to obtain adequate shelf life of the active ingredient in drug products by the preparation and packaging of dry (no water) formulations such as powders for reconstitution, granules and tablets. The dry product is then reconstituted with a solvent, generally water, just prior use. After reconstitution, many of these reconstituted products have very limited shelf life, usually just a few days even if stored in refrigerator. For example, many injectable dosage forms are reconstituted just before administration by adding sterile injectable water.

Solid dosage forms such as tablets that can contain active ingredients that are water labile are often packaged with a desiccant or under a hermetically sealed foil to improve stability.

A significant number of active compounds are subject to decomposition in the presence of water. The chemical mechanisms of composition vary depending on the active compound. Examples of water labile active ingredients include lactams, penicillins, cephalosporins, various esters such as aspirin, benzocain, N-acetyl p-aminophenol, prostaglandin derivatives, indole-3-carbinol (I3C) and derivatives, proteins, peptides, some amides and like. Detailed information about the drug stability is given by Guillory, K. C. and Poust, R. I., “Chemical Kinetics and Drug Stability” in Modern Pharmaceutics, 179-211, Eds. Banker, G. S. and Rhodes, C. T., Third Edition, Marcel Dekker, Inc., New York, 1996.

Water is the most widely used in all phases of manufacturing of pharmaceutical, cosmetic and cosmeceutical preparations. It is considered the universal solvent. Specific grades of water are used for particular applications in concentrations up to 100 percent. Purified water and water for injection are also used for cleaning operations during the manufacture of pharmaceutical products (Ellison et al., Handbook of Pharmaceutical Excipients, Third edition, Kibbe, A. H. Ed, APhA and PhP, 2000, Washington).

Water is also commonly used aqueous excipient in topical products. It is a very effective plasticizer for the stratum corneum, the horny layer of the skin. It hydrates the skin, enhances the permeation of drugs and gives the elasticity to the skin. However, due to the catalytic effects of hydrogen and hydroxyl ions, water is known to start various degradation reactions such as hydrolysis and oxidation.

The patent literature describes some attempts to stabilize unstable compounds in formulations by physically separating the active ingredient from another ingredient that would cause instability of the active compound or the product in general. A German Patent Application DE 4435805-A1 teaches the use of a separately packaged enzyme-based cream to provide enzyme stability and maximum activity. In U.S. Pat. No. 4,823,985, Grollier et al discloses the use of a special dispenser for two constituents of hair coloration. Glaxo, Inc. in a WO 92/17183 patent uses a sequential dosing system. Parab et al. in U.S. Pat. No. 6,019,988 describe the use of a dispensing assembly comprising two compartments that separately contain chemically incompatible active material and permeation enhancer respectively. This way the permeation of the active was enhanced and the stability of the product was assured. Based on the same concept Mo and Frank (U.S. Pat. No. 6,841,574) describe the use of a two compartment system for the stabilization and the delivery of prostaglandin E₁—a highly water labile active ingredient—in a packaged, paired compartment dosage form comprising a sealed actives compartment and a sealed inerts compartment. The prostaglandin E₁ is contained within the actives compartment that does not contain water, but also includes a bulking agent, and optionally a skin penetration enhancer. The inerts compartment is combined with the contents of the actives compartment prior to use.

Stabilization by mean of separation of the active compound(s) from another active compound or from any other ingredient that if combined with the active would cause rapid degradation of the active requires special packaging and is not convenient for the end user.

The present invention provides a method of long term stabilization of water labile active ingredients in water-based products that does not require special packaging and does not require the user to mix, admix or otherwise prepare the product before use.

A typical example of labile compounds in aqueous medium is prostaglandin class of compounds. Prostaglandins are naturally occurring eicosanoic acid derivatives and were first discovered by von Euler [Arch. Exp. Pathol. Pharmakol. 175:78 (1934)]. They exhibit various pharmacological activities. Prostaglandins are usually named by letters and numbers: A1, A2 . . . E1, E2 etc. This nomenclature is based on chemical similarity, not on the similarity of physiological effects. Specific prostaglandins often have contrary pharmacologic functions.

Prostaglandin E₁, PGE₁, [(13E)-(15S)-11alpha, 15-dihydroxy-9-oxoprost-13-enoate], (alprostadil) is a typical example of this class of compounds. It was first isolated from sheep seminal tissue by Bergström et al., Acta Chem. Scand. 16:501 (1962). Prostaglandin E₁ is the theoretical cyclooxygenase metabolite of dihomo-gamma-linolenic acid (DGLA), Because it has a very short biological half-life, it is virtually undetectable in the plasma of normal humans or other animals [Cawelloet. al., J. Urol. 158:1403-1407 (1997].

The pharmacological activities of PGE₁ include vasodilation, hypotension, and anti-platelet behavior. The IC₅₀ of prostaglandin E₁ for the inhibition of ADP-induced human platelet aggregation is 40 nM [Kobzar et. al., Proc. Estonian Acad. Sci. Chem. 40:179-180 (1991)].

In clinics the vasodilating and anti-hypertensive effects of PGE₁ are used to treat male erectile dysfunction (ED) and to provide emergency vasodilation of the patent ductus arteriosus in infants whose cardiac anomalies require pulmonary shunting for survival [Okada et al. Prostaglandins 7:99-106 (1974); Padma et al., N. Engl. J. Med. 336:1-7 (1997); and Olley et al., Annu. Rev. Med. 32:375-3785, (1981)]. For treating ED in human males, the intracavernosal effective dose range for prostaglandin E₁ is reported to be 2- 80 μg, whereas prostaglandin E₁ delivered as a intraurethral suppository uses a dose range of 125 μg-1000 μg. Additional pharmacological activities of alprostadil are reported to be acceleration of cervical ripening during the labor, and inhibition of gastric secretion and the treatment of wound healing.

Alprostadil is an inherently unstable compound. Under various circumstances including temperature, pH, humidity, light etc. after a cascade of reactions it decomposes to mainly prostaglandin A1 [(13E)-(15S)-15-Hydroxy-9-oxoprosta-10,13-dienoate] and if the conditions are extreme such as highly alkaline medium it decomposes to prostaglandin B1 [Lee et al., J. of Chromatogr 555:73-80 (1991)]. In some cases epi-derivative of prostaglandin E₁ can also start to form. Both prostaglandin A1 and B1 are more stable than prostaglandin E₁. However as known both of these compounds are significantly less potent than prostaglandin E₁.

In the presence of water, the key reaction is based on the self-dehydration of alprostadil rather than oxidation or other mechanism. The reaction cascade starts by the loss of water from the side chain of the molecule and introduction of a double bond to the oxocyclopentane moiety with the formation of alcohol group attached to the ring [Teegarden et al., Pharm. Res. 5:482-487 (1988) and ibid. 6:210-215 (1989)].

Prostaglandins trend to be hydrolytically unstable and are very difficult to formulate as storage-stable solutions. Additional attempts were undertaken to stabilize the compound and to obtain formulations with acceptable shelf-life. Adjustment of the pH to lightly acidic conditions was one of the approaches. Although in acidic medium the stability is better than at alkaline pH, in the reality this improvement permits for only for a few weeks stability under refrigerated conditions. Regardless of the pH, alprostadil quickly decomposes to less active compounds in an aqueous environment at normal room temperature and in refrigerated temperatures. [Teegarden et al., Pharm. Res. 5:482-487 (1988); ibid. 6, 210-215 (1989)].

The compound itself has poor solubility in aqueous systems. For example in low acidic medium the solubility is in the range of 10 μg's; in slightly alkaline medium the solubility increases to the range of 100 μg's.

In patent literature, various attempts have been made to stabilize various prostaglandins by different approaches. Stabilization efforts include gelling of the formulation containing the active material with colloidal silicon dioxide after dissolving in a non-aqueous solvent such as described in U.S. Pat. No. 4,680,312 by B. S. Johnson. The use of cyclodextrin derivatives as complexing and stability enhancing agents has also been utilized to stabilize the drug (U.S. Pat. No. 4,054,736). Additional methods include the preparation of lyophilized powders of prostaglandin E₁ by dissolution in a buffered solution of lactose and tertiary butyl alcohol and freeze-drying the resulting composition. The formed lyophilized powders are said to be stable at normal room temperatures but require reconstitution in appropriate solvent (U.S. Pat. No. 5,770,230). Two US Patents assigned to Alcon Laboratories also describe a procedure to obtain storage stable compositions of other prostaglandins in the presence of polyethoxylated castor oils (U.S. Pat. No. 5,631,287 and No. 6,011,062).

An additional example from the patent literature provides an approach to stabilize prostaglandin E₁ in non-aqueous medium for vaginal and rectal delivery using alkali metal salts of fatty acids (Nishimura et al., U.S. Pat. No. 5,491,171).

A recent patent from Drizen et al. claims the utilization of a negatively charged polymer such as hyaluronic acid alone or in combination with nonionic polymers to prepare stable semi-solid formulations of prostaglandin E₁ for the treatment of sexual dysfunction. However, no stability data were presented (U.S. Pat. No. 6,514,536).

Foldvari et al., Int. J. Pharm. Compounding, 3:160-163, (1999) and Foldvari et al. Urology, 52:838-843 (1998) also reported a liposome approach to stabilize the drug. A poster presented at 2003 AAPS meeting (AAPS Meeting abstracts, T 2189, Salt Lake City, USA 2003) by Choi et al. reports the use of completely non-aqueous solvents gelled with hydroxypropyl cellulose to obtain stable and effective prostaglandin E₁ and its ethyl ester.

All the above stabilization approaches have met with varying success but without adequate long term commercial shelf-life these formulations have no practical or commercial utility.

Alprostadil is ineffective after oral administration because it is very unstable in an acidic aqueous environment therefore the method of delivery to the body is by parenteral routes or through the skin or mucous membranes. Commercial formulations of injectable alprostadil are available for the treatment of erectile dysfunction. However, to circumvent water catalyzed instability, and to obtain commercial room temperature shelf life, the active material is lyophilized using a proprietary process and immediately before use the lyophilized formulation is reconstituted with an aqueous buffered solution. The resulting mixture is injected through the side of the penis and into the corpus cavernosum of the penis shortly before intercourse. This approach is utilized by the commercially available products, Edex®, and Caverject®. In the Edex®, product alprostadil is complexed with alpha-cyclodextrin followed by lyophilization to obtain a room temperature stable powder. The Caverject® product utilizes a lyophilized powder containing alprostadil in a base of lactose, sodium citrate, and benzyl alcohol.

Theoretically, it is also possible to protect the active by using a non-aqueous medium. A typical example is Muse®, a commercially available urethral suppository formulated by using polyethylene glycols to stabilize the drug (U.S. Pat. No. 5,474,535 to Place et al.). However, various studies have shown that alprostadil in polyethylene glycol and other non-aqueous media can have improved stability under refrigerated conditions but the release of the drug for therapeutic effect can be severely impaired. The low efficacy of this polyethylene glycol-based penile suppository formulation compared to the intracavernosal injection products is probably due to the absence of any water in the formulation, which can enhance the partitioning of the drug in water lipid interface at the site in the urethra where it is inserted, which is in the proximal urethral, approximately 3 centimeters from the tip of the penis.

Literature and patents show a greatly improved delivery of alprostadil from aqueous bases topical formulations designed to deliver alprostadil through intact skin and into underlying tissues for a therapeutic effect. In-vitro studies have shown that polyethylene glycol-based formulas containing alprostadil applied to intact skin have extremely poor permeation of the active ingredient. These non-water-containing matrices do not readily release a sufficient amount of drug from the matrix to be therapeutically effective. In contrast, similar in-vitro studies have shown that the skin permeation of alprostadil from specially formulated aqueous-based matrices is relatively rapid with therapeutic quantities permeating the skin within minutes of application.

The ability of the active ingredient to penetrate intact skin or mucosal tissue is thus a key to the utility of this type of product. Thus, it is important to protect the therapeutic effectiveness of the active ingredient in the formulation when considering any effort to stabilize an intrinsically unstable active ingredient. The stabilization of prostaglandin E₁ must therefore not impede the delivery of the active drug otherwise the utility of this type of formulation is lost.

In addition, it is also desired to have a convenient and ready to use dosage form that does not require refrigeration or reconstitution before use. Currently, no ready to use aqueous solution, aqueous semisolid or non-aqueous semisolid formulation of alprostadil has been described in patents or literature to be stable at room temperature.

Topical formulations that have been described in patents and literature have all required refrigeration to achieve sufficient long term stability suitable for commercialization or they relay on some level of premixing prior to use. These non-room temperature-stable products contain popular thickening and gelling agents such as Carbomers, povidone derivatives, naturally occurring gums and water. However, these compositions are only stable for longer than one year if stored at refrigerated conditions at 4-8° C. (Buyuktimkin et al., U.S. Pat. No. 6,046,244).

The utilization of a mainly lipid based liposome formulation containing alprostadil where the active ingredient is entrapped in the lipid phase was also devised. This method of stabilization theoretically circumvents the use of water. However, the release of a sufficient amount of active ingredient from this liposomal matrix to result in an adequate therapeutic effect can still be a major challenge. Another, and perhaps more practical way can be the formulation of alprostadil into liposomes and then incorporate these liposomes into other formulations [Foldvari et al., Eur. J. Drug Metab. Pharmacokinet. 22(2):111-120 (1997)]. However due to its low aqueous stability, the alprostadil is entrapped in external lipid layer of the unilamellar vesicle that could severely impede the release of the active ingredient from the formulation and result in unacceptable clinical effect.

Another example of the need for the present invention is the stabilization of L-ascorbic acid. Instability of L-ascorbic acid in aqueous medium is well documented [Ascorbic Acid, Budavari, S. (ed), The Merck Index, 12^{th} edition, p 139, (1996), Whitehouse Station]. Several examples of the stabilization of L-ascorbic acid are cited in the patent literature.

For example, in one US patent, a composition for topical application is provided containing ascorbic acid and at least one polyol, such as propylene glycol and propylene glycols, the latter being present in an effective quantity to obtain a water activity value of the composition which is lower than or equal to 0.85, and at least one structuring agent chosen from polymers and oils, with a view to stabilizing the ascorbic acid. It is claimed that in such a composition, ascorbic acid retains its effectiveness over the course of time. However, no real time data are given to substantiate a commercially viable long term room temperature product. (Cantin et al. U.S. Pat. No. 5,736,567).

Another patent is based on a phosphonic acid derivative and on a metabisulphite for stabilizing ascorbic acid. The invention also relates to compositions, in particular cosmetic and dermatological compositions, containing ascorbic acid and this stabilizing system, and to the use of these compositions in the cosmetics and/or dermatological fields. The inventors claim the stability based on color change of compositions kept for 3 days at 45° C. However, these data are not sufficient for substantiating a commercially viable long term room temperature product. (Nguyen et al. U.S. Pat. No. 6,110,476).

In a further patent, a stable non-aqueous composition is provided to treat and/or prevent photo-aged skin and related skin disorders such as sunburn, wrinkles, poor skin tone and skin discoloration by topically applying to the skin the treatment composition containing an effective amount of a compound such as ascorbic acid, derivatives of ascorbic acid and/or extracts containing ascorbic acid, in a pharmaceutically acceptable vehicle containing a substantially anhydrous base having no water added. The anhydrous base stabilizes the compound, so that the compound remains effective for an effective period of time, even in the presence of exposure to water (Hernandez et al. U.S. Pat. No. 6,110,477).

Another invention relates to a composition containing, in a physiologically acceptable medium, an unstable active agent (L-ascorbic acid) in an oxidizing medium and an oil phase comprising cross-linked elastomer solid organopolysiloxane particles consisting of at least one oxyalkylene group and oxyethylene, in particular. The unstable active agent in an oxidizing medium is stabilized by the use of cross-linked elastomer solid organopolysiloxane particles having an oxyalkylene group for the stabilization of an unstable active agent in an oxidizing medium. The composition can be anhydrous or take the form of an emulsion (Jager-Lezer et al. U.S. Pat. No. 6,544,532). This patent does not provide any stabilization data.

In an other US Patent, a stable, however non-aqueous composition including a water-sensitive pharmacologically active agent, such as an enzyme, an antibiotic or a vitamin, a hydrophilic non-polar primary solvent, an optional hydrophilic non-polar secondary solvent and optional pharmaceutical or cosmetic adjuvants to enhance appearance for topical use is disclosed. Where the water-sensitive or water-degradable component is L-Ascorbic acid and the principal solvent is N-methyl-2-pyrrolidone (“NMP”) the composition is stable for many months at a concentration of L-Ascorbic acid of up to about 40% w/v (Chen et al. U.S. Pat. No. 6,645,508).

Biatry et al. teach a composition containing; preferably in a physiologically acceptable medium comprising an aqueous phase, at least one oxidation-sensitive hydrophilic active principle selected from the group consisting of ascorbic acid and its derivatives and at least one non-crosslinked N-vinylimidazole polymer or copolymer, the active principle and the polymer or copolymer both being in the aqueous phase, useful for preventing and/or treating cutaneous signs of intrinsic ageing (US Published patent application 20030125378). However, only ascorbic acid derivatives were reported to be used in the claims.

A recent patent application publication discloses an oil-in-water emulsion, comprising: (a) one or more interface-active substances A selected from the group consisting of branched glucose derivatives or unbranched alkyl radical having 1 to 24 carbon atoms, (b) one or more interface-active substances, branched or unbranched, saturated or unsaturated fatty acid esters having 8 to 24 carbon atoms and (c) ascorbic acid (Nielsen et al. US Published patent application 20040258646).

A third example of the need for the present invention is the stabilization of a water labile dietary compound indole-3-carbinol (I3C) that shows promising effectiveness in the treatment and prevention of various cancers resulting from a human papilloma virus (HPV) infection.

The effectiveness of dietary indoles mainly I3C, diindolylmethane (DIM) and like in the treatment of human papilloma virus infections associated with high risk types 16, 18, 31, 33, and 45 (cervical dysplasia, cancer of cervix) and low risk types 1-5 (cutaneous warts) 2, 6, 11, 13, 32 (respiratory system warts) is well documented [Jin et al., Cancer Res. 59:3391-3397 (1999); Cassie et al., Chem.-Biol. Interact 102:1-16 (1996); Chang et al., Biochem. Pharmacol. 58:825-834 (1999); Yuan et al., Anticancer Research 19:1673-1680 (1999); Chinni et al., Clin. Cancer. Res. 8:1228-1236 (2002)].

In addition to the effectiveness of dietary indoles on the treatment of HPV related complications, various investigations showed the effectiveness of those indoles in various other cases such as the ability to impede the growth of breast cancer tumors [Wattenberg and Loub, Cancer Res. 38:1410-1415 (1978); and Grubbs et al., Anticancer Res. 15:709-716 (1995)], chemoprevention and treatment of neoplasia (U.S. Pat. No. 6,399,645), adjustment of steroid hormone metabolism as anti-estrogens (U.S. Pat. No. 6,086,915), treatment of premenstrual syndromes and menopause, weight loss promoters (U.S. Pat. No. 6,534,085) and oral absorption enhancing agents (U.S. Pat. No. 6,416,793).

I3C is extremely unstable and it does not completely survive exposure to gastric acid [de Kruif et al. Chem.-Biol. Interact. 80:303-315 (1991)]. I3C is partly converted into several natural indole derivatives with biological activities such as its dimer 3,3′-diindolylmethane (DIM) and indolo[3,2-b]carbazole (ICZ) through an acid-catalyzed reaction occurring in the low-pH environment of the stomach. Other investigators have shown that I3C conversion product DIM was also effective in reducing DMBA-induced mammary tumors, but DIM apparently was not consistently as effective as I3C [Wattenberg and Loub Cancer Res. 38:1410-1415 (1978)].

To enhance the stability, Firestone et al. (U.S. Pat. No. 6,656,963) developed various novel bioactive derivatives of I3C including esters. Their data indicate that I3C itself, and not its acid breakdown products, is a potent anti-tumor agent, and that stable derivatives of I3C can be used to inhibit the growth of estrogen-dependent or independent breast cancer cells and other types of cancer cells that reveal induced CDK6.

A general method for stabilization for aqueous formulations of an interferon, a granulocyte-macrophage colony-stimulating factor or an interleukin are said to be provided by incorporating methionine, histidine or mixtures thereof into the composition (Patel et al. U.S. Pat. No. 5,358,708).

The present invention provides a method for the stabilization of PGE₁, I3C and derivatives, ascorbic acid and other labile compounds, as well as compositions that utilize that method. That method also includes an enhanced delivery system for I3C, DIM, and like.

### BRIEF SUMMARY OF THE INVENTION

A stabilized pharmaceutical composition that comprises a pharmaceutical compound that is labile to degradation in the presence of water is disclosed. That composition contains water, an effective amount of labile pharmaceutical compound and a degradation-inhibiting amount of a C₄-C₁₂ polyol, preferably a C₄-C₆ polyol, that is substantially free of aldehyde or ketone carbonyl groups. The composition exhibits no more than 10 percent degradation of the labile pharmaceutical compound over a period of storage of at least 12 months when stored at ambient room temperature. The C₄-C₁₂ polyol is preferably present in an amount of about 40 to about 70 percent by weight of the composition. A contemplated composition preferably further contains up to 40 weight percent of a solubilizing alcohol that contains up to about 12 carbon atoms, and one or more polymeric thickeners in an amount of up to about 10 weight percent.

A method of stabilizing an aqueous composition containing a pharmaceutical compound that is labile to degradation in the presence of water is also contemplated. That method comprises admixing water, an effective amount of labile pharmaceutical compound, and a degradation-inhibiting amount of a C₄-C₁₂ polyol that is substantially free of aldehyde or ketone carbonyl groups to form a composition. The composition so formed exhibits no more than 10 percent degradation of the labile pharmaceutical compound over a period of storage of at least 12 months when stored at ambient room temperature. The preferences noted above are applicable for use in the preparation of a composition made according to this method.

### DETAILED DESCRIPTION OF THE INVENTION

This invention contemplates a method of stabilization of various pharmaceutical compounds that are unstable in aqueous medium using alprostadil, L-ascorbic acid, and indole-3-carbinol (I3C) as examples. Without wishing to be bound by theory, it is believed that the observed stabilization is achieved by structuring water to the extent that a direct water-drug interaction in monophasic formulation medium is prevented. This stabilization is accomplished by the selection of specific ingredients, concentrations and solution conditions such that water is sufficiently structured to prevent interaction with the water labile portion of the active ingredient molecule. The structuring of water is ultimately accomplished by hydrogen bonding, Van der Waals forces and dipole-dipole interactions.

A composition contemplated by this invention includes a water labile pharmaceutical compound that in the presence of moisture is chemically changed or decomposed at a rate that prevents the composition having utility as a commercial product. The labile compound can have various labile groups and moieties that in the presence of water can either split or polymerize into inactive or toxic compounds or indirectly catalyzed other sets of decomposition reactions such as oxidation. The labile compounds can even add or loose water to form other compounds with quantitatively or qualitatively different pharmacological activities. The present invention provides a method of stabilizing an aqueous composition to degradation of the labile compound such that no more than 10 percent of the labile compound (e.g., drug) is degraded over a period of storage of at least 12 months when stored at ambient room temperature. It is believed that the method provides for structuring water or sequestering molecules resulting in reduced water interactions that result in significantly improved stability of the water labile compound in a water-containing environment.

The specific ingredients used to accomplish adequate stabilization include hydroxylated GRAS compounds that are sugar-related polyalcohols that are substantially free of aldehyde or ketone carbonyl groups. The term “polyol” or “polyalcohol” as used in the present invention denotes a non-volatile compound that has carbon, hydrogen and oxygen atoms that correspond in formula to CₙH₂ₙ₊₂Oₙ, or CₙH₂ₙ₊₂Oₙ₋₁, where “n” is an integer that is 4-12. Thus, the contemplated polyols are C₄-C₁₂ polyols. These materials are C₄-C₁₂ polyols such as erythritol, adonitol, arabitol, xylitol, sorbitol, mannitol, maltitol, lactitol, dulcitol, iditol, and mixtures thereof, are preferably C₄-C₆ polyols such as erythritol, adonitol, arabitol, xylitol, sorbitol, mannitol, dulcitol, iditol and mixtures thereof.

By “substantially free of aldehyde or ketone carbonyl groups” it is meant that a carbonyl group provided by an aldose or ketose sugar can be present as a minor impurity, but typically at less than about 5 percent by weight and more preferably at about one percent or less. Thus, a contemplated composition is typically more than 95 percent free of aldehyde or ketone carbonyl groups provided by an aldose or ketose sugar, and more preferably a composition is more than about 99 percent free of such carbonyl groups.

The amount of C₄-C₁₂ polyol is present in a degradation-inhibiting amount that is typically about 40 to about 70 percent by weight, preferably about 50 to about 67 percent by weight, and most preferably about 53 to 65 weight percent of the composition, to obtain optimum aesthetic quality and drug stability and solubility. To enhance the storage quality it can also be helpful to utilize some sequestrants derivatives in an amount of up to about 1.0 weight percent, and it is preferred to use an amount up to about 0.2 by weight.

In general, a contemplated composition contains one or more labile pharmaceutical compounds (drugs) in an amount of about 0.001 to about 20.0 wt percent. A composition of the present invention also typically includes one or more solubilizing alcohols in an amount up to about 40.0 percent by weight (wt % or percent) preferably in amount of about 5 to about 25 weight percent. It is preferred to use one or more polymeric thickeners such as a modified starch derivative or a polyacrylic acid in an amount of up to about 10 wt percent.

Some labile active materials or agent or compound can have limited solubility in water and therefore can require a surfactant or other appropriate ingredients and co-solvents in the composition. Such co-solvents are typically employed at a concentration up to about 25 of weight percent.

The labile active agent can be present in its neutral, ionic, salt, basic, acidic, natural, synthetic, various crystalline or amorphous, diastereomeric, isomeric, enantiomerically pure, racemic, hydrate, chelate, derivative, analog, or other common form. A therapeutic compound contained within the present system can be incorporated in formulations as their pharmaceutically acceptable salts.

As used herein, “pharmaceutically acceptable salts” refers to derivatives of the above-disclosed compounds wherein especially some physical characteristics or irritation potentials were modified by reacting them with an acid or base as needed to form preferably an ionically bound pair. Illustrative pharmaceutically acceptable salts formed with the organic and inorganic acids include salts using the acids discussed below. Such acids include without limitation inorganic acids such as hydrochloric, hydrobromic, sulfuric, bisulfonic, and phosphoric, and C₁-C₂₄ organic acids such as formic acid, acetic, succinic, citric, isocitric, lactic, maleic, malic, fumaric, cholic, gluconic, glucuronic, pyruvic, oxalacetic, alginic, aspartic, glutamic, pamoic, mucic, D-glutamic, d-camphoric, glutaric, phthalic, tartaric, salicyclic, camphoric, camphorsulfonic, digluconic, methanesulfonic, benzenesulfonic, toluenesulfonic, sorbic, picric, benzoic, cinnamic, and like acids. Mixtures of pharmaceutically acceptable acids are also contemplated. Pharmaceutically acceptable salts formed with pharmaceutically acceptable inorganic and organic bases illustratively include those formed from inorganic bases such as sodium, potassium, calcium or magnesium hydroxides. Illustrative pharmaceutically acceptable organic bases include basic amino acids selected from the group consisting of arginine, lysine, proline, as well as an amine selected from the group consisting of diethanolamine, triethanolamine (Trolamine), trimethylamine, diethylamine, procaine, N,N′-dibenzylethylenediamine, hexamethylenetetramine chloroprocaine, choline, ethylenediamine, and meglumine (N-methylglucamine). Mixtures of pharmaceutically acceptable bases are also contemplated.

A labile pharmaceutical compound generally can be stabilized using the methods described herein. The term “pharmaceutical compound” or “physiologically active agent” is used herein to refer to a broad class of useful chemical and therapeutic agents including labile physiologically active compounds that can benefit from the technology described herein. These compounds can include but are not limited to steroids, antibiotics, antifungal agents, antibacterial agents, antineoplastic agents, analgesics and analgesic combinations, anorexics, anthelmintics, antiarthritics, antiasthia agents, anticonvulsants, antidepressants, antidiabetic agents, antidiarrheals, antihistamines, anti-inflammatory agents, antimigraine preparations, antimotion sickness preparations, antinauseants, antiparkinsonism drugs, antipruritics, antipsychotics, antipyretics, antispasmodics, including gastrointestinal and urinary; anticholinergics, sympathomimetics, xanthine derivatives, cardiovascular preparations including calcium channel blockers, betablockers, antiarrhythmics, antihypertensives, diuretics, in-situ oxygen generating agents, vasodilators including general, coronary, peripheral and cerebral; central nervous system stimulants, cough and cold preparations, decongestants, hormones, enzymes, hypnotics, immunosuppressives, muscle relaxants, parasympatholytics, parasympathomimetics, psychostimulants, sedatives, tranquilizers, allergens, antihistaminic agents, anti-inflammatory agents, physiologically active peptides and proteins, vitamins, cosmeceuticals, nutraceuticals, phytochemical compounds, dietary supplements and derivatives, ultraviolet screening agents, perfumes, insect repellents, hair dyes, and the like. The term “physiologically active” in describing the agents contemplated herein is used in a broad sense to comprehend not only agents having a direct pharmacological effect on the host but also those having an indirect or observable effect which is useful in the medical arts, e.g., the coloring or opacifying of tissue for diagnostic purposes, the screening of U.V. radiation from the tissues and the like.

The above-mentioned list should not be considered exhaustive and are merely exemplary of the many embodiments considered within the scope of the invention. Many other active compounds can be administered with the formulation of the present invention. Additional common examples of general formulation aids, antioxidants, antimicrobials, and chelating agents for the present formulations can be found in Handbook of Pharmaceutical Additives, compiled by Michael and Irene Ash, Gower, Aldershot, UK (1995).

The solubilizing agents include but are not limited to pharmaceutically acceptable shorter chain alcohols containing up to about 20 carbon atoms and derivatives having following structural formula:
CXYZ—C(xy)m—OH
where X,Y,Z,x,y are hydrogen, substituted and unsubstituted alkyl, halogen, amine, amide, alkyl substituted ether and ester and hydroxyl radicals. Illustrative compounds include ethanol, propanol, isopropanol, butanols, pentanols, pantothenols, propylene glycol, ethoxyethoxyethanol, di-propylene glycol, hydroxypropylene glycol, hexylene glycol, 1,3-butylene glycol, 1,2,6-hexanetriol, ethoxylated glycerin, propyloxylated glycerin and like and mixtures thereof. For shorter chain alcohols m is an integer between 1 and 18. A preferred solubilizing alcohol contains up to about 12 carbon atoms and more preferably contains up to about 8 carbon atoms.

A contemplated composition of this invention contains a high concentration of polyalcohol. However, the viscosity of these compositions can be adjusted higher by the additional of thickening agents in amounts of up to about 10 weight percent, and preferably about 0.2 percent w/w to about 10 percent w/w, and more preferably about 0.5 to about 2 weight percent.

Exemplary thickeners are ionic or non-ionic polymers such as hyaluronic acid, chitosan, collagen and derivatives, polyacrylate gums, polyvinyl alcohol; polyvinyl pyrrolidone, naturally occurring polymers such as pectin, gelatin, or gums such as xanthan, carrageenan, karaya, guar, acacia, locust beans gum and various galactomannan and their derivatives, cellulose based gums such as hydroxy propyl cellulose, hydroxy propyl ethyl cellulose, hydroxy propyl methyl cellulose and similar materials, as well as some inorganic materials such as montmorillonite clays, hydrated aluminum silicate, and fumed silica. However food grade chemically and physically modified starches such as hydroxypropyl starch (Zeina B860) and hydroxypropyl phosphate starch (Zeina B862) or guar gum derivatives such as hydroxypropyl guar gum (Jaguar HP120) are preferred choices. The viscosity of a contemplated formulation can also be increased by admixture of up to 10 percent solid sorbitol.

An alternative or addition to the polysaccharide gum is a polyacrylic acid polymer. A common variety of polyacrylic acid polymer is known generically as “Carbomer” that are polyacrylic acid polymers lightly cross-linked with polyalkenyl polyether. There materials are commercially available from the B. F. Goodrich Company (Akron, Ohio) under the designation “CARBOPOL®”. A particularly preferred variety of carbomer are those designated as “CARBOPOL 940” and “CARBOPOL 934”.

Other polyacrylic acid polymers suitable for use in practicing this invention are those commercially available under the designations “Pemulen®” (B. F. Goodrich Company) and “POLYCARBOPHIL” (A. H. Robbins, Richmond, Va.). The Pemulen polymers are copolymers of C₁₀ to C₃₀ alkyl acrylates and one or more monomers of acrylic acid, methacrylic acid or one of their simple esters cross-linked with an allyl ether of sucrose or an allyl ether of pentaerythritol. POLYCARBOPHIL® is a polyacrylic acid cross-linked with divinyl glycol.

The stability of an aqueous composition containing a dissolved or dispersed labile drug can be improved further by preparing the solutions containing C₄-C₁₂ polyol (sorbitol) in an appropriate buffer. Each labile compound can have an optimal pH value range that can be readily determined where such values are not published. Examples of suitable buffering agents include acetic acid, citric acid, carbonic acid, phosphoric acid, boric acid, and tris and derivatives and also the pharmaceutically acceptable salts of the foregoing. Such buffers, if utilized, will be employed in an amount of about 0.001 to about 2% weight.

Although the formulations due to their elevated polyalcohol content and resulting hypertonicity are self-preserving, the incorporation of some antimicrobials can further enhance the shelf-life and stability. Suitable preservatives include: benzalkonium chloride, thimerosal, chlorobutanol, parabens, phenylethyl alcohol, disodium edetate, sorbic acid or other agents known to those skilled in the art. These ingredients provide an additional level of protection, although uncommon, against the decomposition. Such preservatives, if utilized, are used in an effective amount, which typically is an amount of about 0.001 to about 1.0% weight. Suitable examples of sequestrating agents include, but are not limited to, EDTA, sodium citrate, tartaric acid, edetic acid, cyclodextrin derivatives.

Buffer components, if utilized, are typically present up to about 2.0wt %. The composition also comprises the utilization of water in amount up to about 45.0%, and it is especially preferred to use an amount between about 20 and about 35% weight.

Active materials derivatives of this invention can be used in a variety of pharmaceutical preparations. The compositions of this invention can be administered in various pharmaceutical dosage forms. These dosage forms also include immediate release, extended release, controlled release, sublingual, buccal, hypodermic tablets, triturates, powders, dry suspensions, capsules, injectables, suppositories, dispersions,. emulsions, solutions, and in other suitable forms. In these dosage forms various GRAS formulations aids are conveniently processed with active materials to give the desired product. The usage and limitations ingredients utilized in the formulations can be found in “Handbook of Pharmaceutical Ingredients”, Kibbe, A. H. Ed., American Pharmaceutical Association and Pharmaceutical Press, Washington, D.C., Third Edition, 2000. These compounds and compositions are given as examples and should not be limited to these variations. These formulations are manufactured in the light of the present disclosed information and by fabrication techniques well known in the art and set forth in various scholarly and professional publications such as described for example in Introduction to Pharmaceutical Dosage Forms, by Howard C. Ansel, Third Edition, Lea & Fabiger, Philadelphia (1981).

A contemplated composition of the invention can be formulated as a lotion, a cream or a gel. The composition can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. In addition to the above different forms of the composition, the compositions can also be dispensed by any of the known delivery forms, including but not limited to unit dosage or multi-dosage forms and bulk forms. Examples of unit dosage forms include syringes, gel caps, blister packs, and specially designed dispensers. Bulk forms can be stored in tubes, bottles, jars, pumps, aerosol and formed glass containers.

### Mechanism of Stabilization

Based on the formulations described in the Examples hereinafter, the compositions contain approximately 87 percent to 92 percent (w/w basis) 70% aqueous sorbitol. These values correspond to approximately 61 percent and 64 percent sorbitol and approximately 26 percent and 28 percent water.

Theoretically, in equimolar basis 1 molecule of sorbitol (mw 182.17) corresponds to 1 molecule of water (mw 18.02). For example a 100 g of formulation containing 61.2 g of pure sorbitol corresponds to 0.34 moles of material, whereas in 26.2 g water there are approximately 1.45 moles of water. Therefore sorbitol/water molar ratio is approximately 1:4.4.

Based on the above calculation each sorbitol molecule should structure and hold 4 to 5 molecules of water, which is highly possible because the structure contains 4 secondary alcohol groups at 2, 3, 4, and 5 positions and two primary alcohol groups at 1 and 6 positions of the molecule. The formulation composition, as it is, is highly capable of holding even larger amounts of water thus reducing the possibility of interaction with the water labile active ingredient.

A supporting assumption is based on the stability of the same formulations in the presence of 70% disaccharide sucrose (mw 342.3) solution. Room temperature (RT) studies showed that the stability was not as good as with sorbitol. As given in Example 5 at room temperature after 16 and 28 days 1.20 and 3.07 percents of PGA1 were formed respectively, whereas at the same period of time sorbitol-containing formulations did not show any decomposition (Table 1).

The sucrose-containing composition had 0.19 moles of sucrose versus 1.6 moles of water. Although the sucrose molecule has 5 secondary alcohol groups and three primary alcohol groups, it is believed that because the lower amount of sucrose molecules available (nearly as half as sorbitol) there is not sufficient hydrogen bonding groups available, so the stability of the formulation is impaired. In addition, sucrose is a lactone whereas sorbitol is a straight chain hexahydric aliphatic alcohol therefore water binding capacity of the former is lower. Based on these assumptions, simple water-soluble sugars and polyalcohols such as xylitol, erythritol can also be utilized as a substitute for sorbitol.

The selection of sorbitol as water binding agent was mainly based to few criteria. The compound has GRAS status and has a long history of utilization in a variety of formulations and has an established safety record. It also has a large water holding capacity. Because it is not a polymer, all carbon atoms of the molecule contain hydroxyl groups whereas with other carbohydrate polymers this capacity is reduced due to the attachment of other group chains to the structure.

Another advantage of the use of sorbitol is its ability to form complexes with di- and trivalent cations, by reducing the possibility of their catalytic oxidation enhancing properties. In addition, due to the hypertonicity of the sorbitol bacterial growth is impaired.

For the present invention various sugar-based, sugar related polyalcohol (sucrose, glucose, sorbitol (D-glucitol), erythritol, and xylitol etc.) formulations were prepared and evaluated for physical and chemical stability. Unexpectedly, selected formulations showed excellent stability after long-term storage at room temperature. Those compositions contained a C₄-C₁₂ polyol at the above-described concentration.

The following provides some illustrative compositions of the formulations examined. These examples are illustrative of the compositions of the present invention and are not to be considered as limiting.

| | COMPOSITION AND EXAMPLE NUMBER | | | | | | | | |
|---|---|---|---|---|---|---|---|---|---|
| | 1 | 2 | 3 | 4 | 5 | 6 | 7 | 8 | 9 |
| INGREDIENT | WEIGHT PERCENT OF TOTAL COMPOSITION | | | | | | | | |
| Alprostadil USP | 0.1 | 0.1 | 0.1 | 0.1 | 0.1 | — | — | — | — |
| Ethanol, 200 proof | 5 | — | 3 | — | 5 | — | — | — | — |
| USP | | | | | | | | | |
| Diethylene Glycol | — | 5 | — | 5 | — | — | — | 10 | 10 |
| Monoethyl Ether | | | | | | | | | |
| NF | | | | | | | | | |
| Hydroxypropyl | — | — | 3.0 | 3.0 | — | — | — | — | — |
| Guar gum | | | | | | | | | |
| (Jaguar HP102) | | | | | | | | | |
| Hydroxypropyl | 7.5 | 7.5 | — | — | 7.5 | — | — | 6.5 | 6.5 |
| starch, FCC | | | | | | | | | |
| (Zeina B860) | | | | | | | | | |
| L-Ascorbic acid | — | — | — | — | — | 5 | 5 | — | — |
| I3C | — | — | — | — | — | — | — | 1 | 1 |
| Polycarbophil USP | — | — | — | — | — | — | — | 1 | 1 |
| Lysine USP | — | — | — | — | — | — | — | 1 | 1 |
| Hydroxypropyl - | — | — | — | — | — | — | — | 5 | 5 |
| cyclodextrin | | | | | | | | | |
| Injectable distilled | — | — | — | — | — | — | Qs | — | 75.5 |
| water | | | | | | | 100 | | |
| Sucrose, 70% | — | — | — | — | Qs | — | — | — | — |
| Solution | | | | | 100 | | | | |
| Sorbitol, 70% | Qs | Qs | Qs | Qs | — | Qs | — | 75.5 | — |
| solution USP | 100 | 100 | 100 | 100 | | 100 | | | |

As shown in the table above, some formulations contain alcohol to help the initial solubilization and permeation of the active ingredient. During storage the formulations were protected from light and excessive humidity.

General laboratory scale procedure for the preparation of the formulation was as follows:
1. Active materials and water-soluble ingredients were dissolved or dispersed in organic solvent or in portion of 70% sorbitol solution.
2. Other ingredients were added to the remaining of 70% sorbitol (or sucrose) solution or water and mixed thoroughly.
3. The contents of the items from step 1 were transferred to item 2 and mixed until homogenous system was obtained.

### Stability Studies

The stability of the formulations was examined by HPLC and the evaluation was followed by comparison to an appropriate reference standard. The sample preparations were performed using established methods. Alprostadil and its primary decomposition products, prostaglandin Al and prostaglandin B1, were also followed using commercially available reference standards.

The stability profiles are given in the following table.

TABLE 1
Stability of Alprostadil from Example 2 and Example 5
at uncontrolled RT (approximately 25° C.)

| | | |
|---|---|---|
| Time interval | Alprostadil % | PGA1 (% w/w) |
| (days) | ±SD | ±SD |

Example 2

| | | |
|---|---|---|
| 16 | 100.0 ± 4.76 | Not detected |
| 28 | 108.03 ± 0.68 | Not detected |
| 208 | 100.70 ± 2.57 | 0.72 ± 0.03 |
| 389 | 105.05 ± 3.32 | 0.66 ± 0.06 |

Example 5

| | | |
|---|---|---|
| 16 | 103.07 ± 1.63 | 1.20 ± 0.024 |
| 28 | 100.31 ± 3.66 | 3.07 ± 0.08 |

The stability of a labile compound in a water-containing environment was further examined by using L-ascorbic acid as a model compound. At present, no liquid or semi-solid L-ascorbic acid composition has been described in patents or literature that contains at least 15% water and has an established 2-year RT shelf-life. For this purpose, a formulation of L-ascorbic acid in 70% sorbitol USP was prepared and kept at uncontrolled room temperature (approximately 25° C.), not exposed to light in a capped clear glass container. No special precaution was taken to hermetically seal the container. Head-space of the container was about ⅓ of the total volume. A pure aqueous control formulation was prepared by substituting 70% sorbitol USP with freshly boiled injectable sterile water USP. It was stored under identical conditions to the storage of the formulation containing 70% sorbitol USP. The compositions of both formulations are given in the table above for examples 6 and 7.

TABLE 2
The stability data of Examples 6, 7

| | % Remaining | | |
|---|---|---|---|
| Formulation | 0 hour | 1 month | 27 months |
| Example 6 (70% | 100.3 | 98.38 | 95.86 |
| sorbitol) | | | |
| Example 7 | 100.4 | 77.88 | 34.43 |
| (Control) | | | |

The assay of L-ascorbic acid in formulations was performed using iodine solution as titrant and starch as indicator. A 70% sorbitol USP solution was also used as a blank control. During the 27 months of storage both the 70% sorbitol solution with the L-ascorbic acid and the 70% sorbitol blank control solution remained clear without any precipitation and with only a slight yellowish tint, however the color of Example 7 without the 70% sorbitol gradually changed to brown.

Other semi-solid formulations of L-ascorbic acid in 70% sorbitol USP in the presence of additional formulation ingredients such as modified galctomannan (guar) gums, modified food starches as consistency modulating agents for semi-solids or other common excipients exhibited similar long term stability behavior.

I3C formulation (Example 8) did not show any change in coloration or precipitation during the 12 months time period at room temperature, whereas water based control formulation (Example 9) showed signs of decomposition as evidence by severe darkening of the solution and precipitation.

Each of the patents and articles cited herein is hereby incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. A stabilized pharmaceutical composition comprising a pharmaceutical compound that is labile to degradation in the presence of water, said composition containing water, an effective amount of labile pharmaceutical compound and a degradation-inhibiting amount of a C₄-C₁₂ polyol that is substantially free of aldehyde or ketone carbonyl groups, said composition exhibiting no more than 10 percent degradation of the labile pharmaceutical compound over a period of storage of at least 12 months when stored at ambient room temperature.

2. The stabilized composition according to claim 1 wherein said C₄-C₁₂ polyol is present in an amount of about 40 to about 70 percent by weight of the composition.

3. The stabilized composition according to claim 1 wherein said composition contains up to 40 weight percent of a solubilizing alcohol.

4. The stabilized composition according to claim 1 wherein said composition contains one or more polymeric thickeners in an amount of up to about 10 weight percent.

5. The stabilized composition according to claim 1 wherein said C₄-C₁₂ polyol is selected from the group consisting of erythritol, adonitol, arabitol, xylitol, sorbitol, mannitol, maltitol, lactitol, dulcitol, iditol and mixtures thereof.

6. The stabilized composition according to claim 1 wherein said C₄-C₁₂ polyol is a C₄-C₆ polyol.

7. A stabilized pharmaceutical composition comprising a pharmaceutical compound that is labile to degradation in the presence of water, said composition containing
water,
an effective amount of labile pharmaceutical compound,
about 40 to about 70 percent by weight C₄-C₆ polyol that is substantially free of aldehyde or ketone carbonyl groups,
up to 40 weight percent of a solubilizing alcohol, and
one or more polymeric thickeners in an amount of up to about 10 weight percent,
said composition exhibiting no more than 10 percent degradation of the labile pharmaceutical compound over a period of storage of at least 12 months when stored at ambient room temperature.

8. The stabilized composition according to claim 7 wherein said C₄-C₆ polyol is selected from the group consisting of erythritol, adonitol, arabitol, xylitol, sorbitol, mannitol, dulcitol, iditol and mixtures thereof.

9. The stabilized composition according to claim 7 wherein said C₄-C₆ polyol is present at about 50 to about 67 percent by weight.

10. The stabilized composition according to claim 7 wherein said solubilizing alcohol contains up to about 12 carbon atoms.

11. A stabilized pharmaceutical composition comprising a pharmaceutical compound that is labile to degradation in the presence of water, said composition containing
water,
an effective amount of labile pharmaceutical compound,
about 50 to about 67 percent by weight C₄-C₆ polyol that is selected from the group consisting of erythritol, adonitol, arabitol, xylitol, sorbitol, mannitol, dulcitol, iditol and mixtures thereof,
up to 40 weight percent of a solubilizing alcohol that contains up to about 12 carbon atoms, and
one or more polymeric thickeners in an amount of up to about 10 weight percent,
said composition exhibiting no more than 10 percent degradation of the labile pharmaceutical compound over a period of storage of at least 12 months when stored at ambient room temperature.

12. The stabilized composition according to claim 10 wherein said C₄-C₆ polyol is sorbitol.

13. The stabilized composition according to claim 10 wherein a polymeric thickener is present in an amount of about 0.5 to about 2 weight percent.

14. A method of stabilizing an aqueous composition containing a pharmaceutical compound that is labile to degradation in the presence of water that comprises admixing water, an effective amount of labile pharmaceutical compound, and a degradation-inhibiting amount of a C₄-C₁₂ polyol that is substantially free of aldehyde or ketone carbonyl groups to form a composition, said composition exhibiting no more than 10 percent degradation of the labile pharmaceutical compound over a period of storage of at least 12 months when stored at ambient room temperature.

15. The method according to claim 14 wherein said C₄-C₁₂ polyol is present in an amount of about 40 to about 70 percent by weight of the composition.

16. The method according to claim 14 wherein said composition further contains up to 40 weight percent of a solubilizing alcohol.

17. The method according to claim 14 wherein said composition further one or more polymeric thickeners in an amount of up to about 10 weight percent.

18. The method according to claim 14 wherein said C₄-C₁₂ polyol is a C₄-C₆ polyol.

19. The method according to claim 18 wherein said C₄-C₆ polyol is selected from the group consisting of erythritol, adonitol, arabitol, xylitol, sorbitol, mannitol, dulcitol, iditol and mixtures thereof.

20. The method according to claim 18 wherein said C₄-C₆ polyol is sorbitol.
